(19) United States
(12) Patent Application Publication
(10) Pub. No.: US 2022/0313737 A1
(43) Pub. Date: Oct. 6, 2022

(54) CD52-DEFICIENT CELLS FOR ADOPTIVE CELL THERAPY

(71) Applicant: CELYAD ONCOLOGY S.A., Mont-Saint-Guibert (BE)

(72) Inventors: Simon BORNSCHEIN, Mont-Saint-Guibert (BE); Eytan BREMAN, Mont-Saint-Guibert (BE)

(21) Appl. No.: 17/619,158

(22) PCT Filed: Jun. 18, 2020
(86) PCT No.: PCT/EP2020/067022

§ 371 Date: Dec. 14, 2021

(30) Foreign Application Priority Data
Jun. 18, 2019 (EP) 19181070.4

(51) Int. Cl. A61K 35/17; C12N 15/113; C12N 5/0783; C07K 14/725; A61P 35/00; C12N 15/11; C12N 9/22; C12N 15/90

(52) U.S. Cl. CPC A61K 35/17; C12N 15/1138; C12N 5/0636; C07K 14/7051; A61P 35/00; C12N 15/11; C12N 9/22; C12N 15/907; C12N 2310/14; C12N 2510/00; C12N 2310/531; C12N 2310/20

(57) ABSTRACT

The present application relates to the field of immunotherapy, more particularly to the field of adoptive cell therapy (ACT). Here, shRNAs designed to downregulate CD52 are proposed. Also proposed are polynucleotides, vectors encoding the shRNA and cells expressing such shRNAs, alone or in combination with a chimeric antigen receptor (CAR). These cells are particularly suitable for use in immunotherapy, especially in combination with immunosuppressive therapies directed against CD52, as is particularly envisaged in allogeneic therapy. The invention provides methods of increasing the efficacy of a T cell therapy in a patient in need thereof. Further, strategies to treat diseases such as cancer using these cells are also provided. The engineered immune cells, such as T-cells or natural killer (NK) cells, expressing such CARs are particularly suitable for treating lymphomas, multiple myeloma and leukemia.

### FIELD OF THE INVENTION

The present application relates to the field of immunotherapy, more particularly to the field of adoptive cell therapy (ACT). Here, shRNAs designed to downregulate CD52 are proposed. Also proposed are polynucleotides, vectors encoding the shRNA and cells expressing such shRNAs, alone or in combination with a chimeric antigen receptor (CAR). These cells are particularly suitable for use in immunotherapy, especially in combination with immunosuppressive therapies directed against CD52, as is particularly envisaged in allogeneic therapy. The invention provides methods of increasing the efficacy of a T cell therapy in a patient in need thereof. Further, strategies to treat diseases such as cancer using these cells are also provided. The engineered immune cells, such as T-cells or natural killer (NK) cells, expressing such CARs are suitable for treating lymphomas, multiple myeloma and leukemia, but other tumors can be treated as well, depending on the specificity of the CAR.

### BACKGROUND

The recent FDA approval of the first two CAR-T therapies, both directed against the B cell antigen CD19, has led to an ever-increasing interest in the CAR-T field. CARs are artificial antigen binding receptors, engineered to recognize cancer antigens through their antigen binding domain, resulting in the activation of the CAR cell through the intracellular signaling domain of the receptor.

Autologous CAR therapies rely on the custom-made manufacturing of therapeutic products from each individual patient's own T cells. Such patient-specific autologous paradigm is a significant limiting factor in the large-scale deployment of CAR technology as it requires significant investment and cannot be upscaled. Moreover, delays inherent to the generation of a therapeutic product preclude immediate administration, thus prejudicing favorable outcomes for the most critically ill patients. Often custom-made autologous product generation may not be feasible for patients who are profoundly lymphopenic owing to previous chemotherapy. Allogeneic therapy, which makes use of cells of healthy donors, has been suggested as an alternative. However, these cells typically need to be modified so they do not attack the patient (Graft versus Host disease or GvHD), and don't engraft or persist long in the patient's body because of rejection mechanism (Host versus Graft response, or HvG). GvHD is typically prevented by impairing or reducing endogenous TCR expression or signalling (see e.g. U.S. Pat. No. 9,181,527), and if acute GvHD develops, it is typically treated with steroids.

Controlling the host response in HvG cannot be done by modifying the patient's cells. To avoid an immediate rejection, the patients are typically lymphodepleted, e.g. using fludarabine, cyclophosphamide, and/or CD52 therapy. Recent studies have shown that preconditioning a patient with immunosuppressive chemotherapy drugs prior to CAR-T cell treatment can increase the effectiveness of the transplanted cells. Alemtuzumab (also known as Campath-1H) is a recombinant, humanized, monoclonal immunoglobulin IgG1 kappa, directed against CD52. Treatment with alemtuzumab induces antibody-dependent cellular cytotoxicity (ADCC) and complement-dependent cytotoxicity (CDC) of CD52-expressing cells. CD52 is present on the surface of mature lymphocytes, such as T cells, B cells and to lower extend on NK cells, but not on stem cells.

However, since CD52 is present on the surface of T cells, anti-CD52 therapy concurrent or overlapping with adoptive cell therapy with T cells will also kill off the therapeutic cells. Thus, there is a continuous need for improvement of persistency of CAR-T cells in the patient in order to ensure long-term durability of the treatment.

Gene editing by knocking out TCR and CD52 has been proposed as a solution (Poirot et al., Cancer Res. 2015; 75(18):3853-64). However, this is not very efficient, as only 12% of the cells achieve a double KO. Further, there is a safety concern in that multiplex gene editing can lead to translocations between double-strand break sites. Such events were detected at a rate of 1×10⁻⁴−2×10⁻² in the study of Poirot et al. Although no proliferative advantage was detected in T-cells that underwent translocation, thorough transformation analysis is needed to ensure the safety of gene-edited CAR-T therapies (Jung and Lee, Mol Cells. 2018; 41(8): 717-723). Given this risk, it is a disadvantage that gene editing is non-reversible and expression cannot be modified over time. It is known that CD52 plays a role in cell mobility and has an anti-adhesion effect. The effect of permanent CD52 knockdown on mobility of therapeutic cells has not yet been addressed. However, non-permanent solutions typically don't achieve sufficient knockdown of CD52 to generate therapeutic benefit, as cells would be killed by parallel anti-CD52 treatment.

Accordingly, there is a need for a therapy that can improve persistence of cells by achieving sufficient CD52 knockdown in a more efficient way, and are more efficient and safe than the present solutions.

### SUMMARY

In the current application, we compared and assessed different methods to generate CD52-deficient cells. MiRNA-based shRNAs allow for co-expression of the shRNA targeting CD52 and the CAR from a single expression vector. Different CD52 targeting shRNAs were assessed for their ability to decrease CD52 surface expression. The shRNAs were expressed from a viral vector, encoding in a first instance only a tCD19 reporter gene and the miRNA-based shRNA scaffold. The efficiency of shRNA knockdown was assessed in primary T cells, isolated from different donors. The most efficient shRNA consistently decreased CD52 expression and did not show any donor variability.

Furthermore, we demonstrate that the co-expression of a miRNA-based shRNA and a CAR from the same viral vector did not affect the activity or expression of the CAR itself.

Most surprisingly, expression of a CD52 shRNA decreased cell surface expression of CD52 in primary T cells to comparable levels as CD52 genome-edited T cells. This unexpected result was further confirmed in functional assays, showing that RNAi-induced downregulation was sufficient to prevent complement mediated cytotoxicity, induced by an anti-CD52 antibody.

The use of shRNA instead of gene editing means no permanent genomic modification is made, eliminating the risk of translocations. Further, shRNA can be put under control of inducible promoters, meaning that the expression can be temporally regulated. Another advantage is that shRNAs are quite small and can be easily combined with other methods, meaning the methods are more efficient. For instance, combined TCR knockdown and CD52 knockdown using shRNA can be achieved with one transduction, which is not feasible with gene editing methods.

Accordingly, provided herein are engineered cells comprising:
A first exogenous nucleic acid molecule encoding a protein of interest;
a second nucleic acid molecule encoding at least a RNA interference molecule directed against CD52.

Typically, the cells are engineered immune cells. According to particular embodiments, the immune cell is selected from a T cell, a NK cell, a NKT cell, a stem cell, a progenitor cell, and an iPSC cell.

According to particular embodiments, the protein of interest is a receptor, particularly a chimeric antigen receptor or a TCR. Chimeric antigen receptors or TCRs can be directed against any target, typical examples include CD19, CD20, CD22, CD30, BCMA, B7H3, B7H6, NKG2D, HER2, HER3, GPC3, but many more exist and are also suitable.

According to particular embodiments, the first and second nucleic acid molecule are present in one vector, such as a eukaryotic expression plasmid, a mini-circle DNA, or a viral vector (e.g. derived from a lentivirus, a retrovirus, an adenovirus, an adeno-associated virus, and a Sendai virus).

According to particular embodiments, the RNA interference molecule is a shRNA molecule. More particularly, the RNA interference molecule is a miRNA molecule. Even more particularly, the miRNA molecule comprises a miR-196 scaffold sequence, preferably a miR-196a2 scaffold sequence.

According to particular embodiments, the RNA interference molecule is under control of a promoter selected from Pol II promoter, a Pol III promoter, a CMV promoter, a U6 promoter, an EF1α promoter (core or full length), a PGK promoter, a CAG promoter, a UBC promoter, a SFFV promoter, a MSCV LTR, a SV40 promoter, GALV LTR and a tRNA promoter.

According to further particular embodiments, the promoter is inducible.

According to particular embodiments, the engineered cell has been further engineered to reduce or inactivate TCR signalling. According to specific embodiments, TCR signalling has been inactivated through gene editing. According to alternative specific embodiments, TCR signalling has been reduced through RNA interference. According to further specific embodiments, the RNA interference to reduce TCR signalling comprises a RNA interference molecule directed against a TCR receptor complex subunit (such as e.g. CD247 or the TCR alpha chain).

According to a further aspect, the engineered cell as described herein is provided for use as a medicament. Particularly, the cell as described herein is provided for use in the treatment of cancer.

This is equivalent as stating that methods of treating cancer are provided, comprising administering to a subject in need thereof a suitable dose of cells as described herein, thereby improving at least one symptom.

According to alternative embodiments, the cells as described herein are provided for use in the treatment of infectious disease, e.g. viral infection. According to alternative embodiments, the cells are provided for use in the treatment of autoimmune disease.

This is equivalent as stating that methods of treating infectious disease are provided, comprising administering to a subject in need thereof a suitable dose of cells as described herein, thereby improving at least one symptom. Or, alternatively, that methods of treating autoimmune disease are provided, comprising administering to a subject in need thereof a suitable dose of cells as described herein, thereby improving at least one symptom.

The engineered cells may be autologous immune cells (cells obtained from the patient) or allogeneic immune cells (cells obtained from another subject), with the latter being particularly envisaged.

### BRIEF DESCRIPTION OF THE FIGURES

FIG. 1: Screening of different CD52 targeting shRNAs A) Percentage of transduced (CD19+) CD4+ or CD8+ T cells are shown, gated on FSC/SSC, viable, CD3+ cells. B) CD52 Mean Fluorescence intensity (MFI) is shown for transduced CD4+ or CD8+ T cells. C) Histogram showing CD52 expression of CD19+CD3+ cells.

FIG. 2: CD52 knockdown in different donors. CD52 MFI is shown for T cells derived from three different donors. Cells were transduced with Mock or CD52 shRNA-3 expressing vector.

FIG. 3: Screening of gRNAs for the generation of CD52 knockout T cells. CD52 MFI is shown for CD4+ and CD8+ T cells at harvest (day 8). For Mock (truncated CD19) and shRNA condition, the gating was performed on CD19+ cells, whereas for the other conditions gating was performed on CD3+ T cells.

Histogram shows the CD52 expression for the three different gRNAs compared to Cas9 only control.

FIG. 4: A) Illustration of the CAR expression vector (e.g. BCMA CAR) without (top) or with (bottom) an integrated miRNA scaffold, allowing for the co-expression of a CAR and a shRNA. LTR: Long terminal repeat; pack. ψ: psi packaging signal; tCD34: truncated CD34, a marker protein; CD52: shRNA against CD52. B) Cells transduced with the different expression constructs were stained with BCMA-Fc fusion protein. In a second step, cells were stained with PE-conjugated anti-Fc and an APC-conjugated anti-CD34 antibodies. MFI of BCMA-Fc staining is shown for CD34+ T cells.

FIG. 5: Different BCMA expressing cancer cell lines were co-cultured with Mock (tCD34), BCMA-CAR expressing T cells or BCMA-CAR-CD52 shRNA expressing T cells. 24 h after co-culture with different BCMA-positive cancer cell lines (RPMI-8226, U266, OPM-2), IFN-γ levels were measured in the supernatants by ELISA.

FIG. 6: BCMA CAR with shRNA against CD52. A) CD52 expression levels (MFI) are shown for Mock (tCD34), BCMA-CAR expressing T cells or BCMA-CAR-CD52 shRNA expressing T cells. One subgroup of T cells was nucleofected with aruncate ribonucleoprotein complex of Cas9 protein and a CD52 gRNA. B) shows an in vitro functional assay of CD52-deficient T cells. In order to assess killing by the alemtuzumab antibody, T cells were treated with 30% complement in the presence of 50 μg/mL anti-CD52 antibody (alemtuzumab) or IgG control antibodies. Number of cells was assessed after 4 h and normalized to a non-treated condition.

FIG. 7: Multiplexing of CD52 and CD3zeta (CD247) creates an all in one vector that does not impact efficacy (of shRNA or the CAR). A) A BCMA CAR including shRNAs against CD247 and CD52, was compared to gRNA CRISPR/Cas9 control. Dotplots showing TCR and CD52 expression are depicted before and after TCR depletion (Top and bottom respectively). B) In vitro functional assay of CD52-deficient T cells. In order to assess killing by the alemtuzumab antibody, T cells were treated with 30% complement in the presence of 50 μg/mL anti-CD52 antibody (alemtuzumab) or IgG control antibodies. Number of cells was assessed after 4 h and normalized to a non-treated condition. C) CAR efficacy was assessed in co-culture with different cancer cell lines. The following CAR T cells were assessed: BCMA, BCMA with shRNA against CD247, BCMA CAR with shRNA against CD52, BCMA multiplex (both shRNA).

### DETAILED DESCRIPTION

### Definitions

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The following terms or definitions are provided solely to aid in the understanding of the invention.

Unless specifically defined herein, all terms used herein have the same meaning as they would to one skilled in the art of the present invention. Practitioners are particularly directed to Green and Sambrook, Molecular Cloning: A Laboratory Manual, 4th ed., Cold Spring Harbor Laboratory Press, New York (2012); and Ausubel et al., Current Protocols in Molecular Biology (up to Supplement 114), John Wiley & Sons, New York (2016), for definitions and terms of the art. The definitions provided herein should not be construed to have a scope less than understood by a person of ordinary skill in the art.

An “engineered cell” as used herein is a cell that has been modified through human intervention (as opposed to naturally occurring mutations).

The phrase “nucleic acid molecule” synonymously referred to as “nucleotides” or “nucleic acids” or “polynucleotide” refers to any polyribonucleotide or polydeoxyribonucleotide, which may be unmodified RNA or DNA or modified RNA or DNA. Nucleic acid molecules include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

A “chimeric antigen receptor” or “CAR” as used herein refers to a chimeric receptor (i.e. composed of parts from different sources) that has at least a binding moiety with a specificity for an antigen (which can e.g. be derived from an antibody, a receptor or its cognate ligand) and a signaling moiety that can transmit a signal in an immune cell (e.g. a CD3 zeta chain. Other signaling or cosignaling moieties can also be used, such as e.g. a Fc epsilon RI gamma domain, a CD3 epsilon domain, the recently described DAP10/DAP12 signaling domain, or domains from CD28, 4-1BB, OX40, ICOS, DAP10, DAP12, CD27, and CD2 as costimulatory domain). A “chimeric NK receptor” is a CAR wherein the binding moiety is derived or isolated from a NK receptor.

A “TCR” as used herein refers to a T cell receptor. In the context of adoptive cell transfer, this typically refers to an engineered TCR, i.e. a TCR that has been engineered to recognize a specific antigen, most typically a tumor antigen. An “endogenous TCR” as used herein refers to a TCR that is present endogenously, on non-modified cells (typically T cells). The TCR is a disulfide-linked membrane-anchored heterodimeric protein normally consisting of the highly variable alpha (α) and beta (β) chains expressed as part of a complex with the invariant CD3 chain molecules. The TCR receptor complex is an octomeric complex of variable TCR receptor α and β chains with the CD3 co-receptor (containing a CD3γ chain, a CD3δ chain, and two CD3ε chains) and two CD3ζ chains (aka CD247 molecules). The term “functional TCR” as used herein means a TCR capable of transducing a signal upon binding of its cognate ligand. Typically, for allogeneic therapies, engineering will take place to reduce or impair the TCR function, e.g. by knocking out or knocking down at least one of the TCR chains. An endogenous TCR in an engineered cell is considered functional when it retains at least 50%, at least 60%, at least 70%, at least 75%, at least 80%, or even at least 90% of signalling capacity (or T cell activation) compared to a cell with endogenous TCR without any engineering. Assays for assessing signalling capacity or T cell activation are known to the person skilled in the art, and include amongst others an ELISA measuring interferon gamma. According to alternative embodiments, an endogenous TCR is considered functional if no engineering has taken place to interfere with TCR function.

A “transmembrane domain” or “TM domain” as used herein is any membrane-spanning protein domain. Most typically, it is derived from a transmembrane protein. However, it can also be artificially designed. Transmembrane domains used herein will typically associate with other transmembrane domains, through charged and non-charged interactions.

The term “signaling domain” as used herein refers to a moiety that can transmit a signal in a cell, particularly in an immune cell. The signaling domain typically comprises a domain derived from a receptor that signals by itself in immune cells, such as the T Cell Receptor (TCR) complex or the Fc receptor. Additionally, it may contain a costimulatory domain (i.e. a domain derived from a receptor that is required in addition to the TCR to obtain full activation, or the full spectrum of the signal in case of inhibitory costimulatory domains, of T cells). The costimulatory domain can be from an activating costimulatory receptor or from an inhibitory costimulatory receptor.

The term “immune cells” as used herein refers to cells that are part of the immune system (which can be either the adaptive or the innate immune system). Immune cells as used herein are typically immune cells that are manufactured for adoptive cell transfer (either autologous transfer or allogeneic transfer). Many different types of immune cells are used for adoptive therapy and thus are envisaged for use in the methods described herein. Examples of immune cells include, but are not limited to, T cells, NK cells, NKT cells, lymphocytes, dendritic cells, myeloid cells, stem cells, progenitor cells or iPSCs. The latter three are not immune cells as such, but can be used in adoptive cell transfer for immunotherapy (see e.g. Jiang et al., Cell Mol Immunol 2014; Themeli et al., Cell Stem Cell 2015). Typically, while the manufacturing starts with stem cells or iPSCs (or may even start with a dedifferentiation step from immune cells towards iPSCs), manufacturing will entail a step of differentiation to immune cells prior to administration. Stem cells, progenitor cells and iPSCs used in manufacturing of immune cells for adoptive transfer (i.e., stem cells, progenitor cells and iPSCs or their differentiated progeny that are transduced with a CAR as described herein) are considered as immune cells herein. According to particular embodiments, the stem cells envisaged in the methods do not involve a step of destruction of a human embryo.

Particularly envisaged immune cells include white blood cells (leukocytes), including lymphocytes, monocytes, macrophages and dendritic cells. Particularly envisaged lymphocytes include T cells, NK cells and B cells, most particularly envisaged are T cells. In the context of adoptive transfer, note that immune cells will typically be primary cells (i.e. cells isolated directly from human or animal tissue, and not or only briefly cultured), and not cell lines (i.e. cells that have been continually passaged over a long period of time and have acquired homogenous genotypic and phenotypic characteristics). According to specific embodiments, immune cells will be primary cells (i.e. cells isolated directly from human or animal tissue, and not or only briefly cultured) and not cell lines (i.e. cells that have been continually passaged over a long period of time and have acquired homogenous genotypic and phenotypic characteristics). According to alternative specific embodiments, the immune cell is not a cell from a cell line.

“Isolated” as used herein means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if such composition is not part of the native environment of the nucleic acid, peptide, or protein. The term also embraces nucleic acids, peptides and proteins prepared by recombinant expression in a host cell as well as chemically synthesized nucleic acids. An “isolated” antibody or antigen-binding fragment, as used herein, is intended to refer to an antibody or antigen-binding fragment which is substantially free of other antibodies or antigen-binding fragments having different antigenic specificities (for instance, an isolated antibody that specifically binds to BCMA is substantially free of antibodies that specifically bind antigens other than BCMA). An isolated antibody that specifically binds to an epitope, isoform or variant of BCMA may, however, have cross-reactivity to other related antigens, for instance from other species (such as BCMA species homologs).

A “vector” is a replicon, such as plasmid, phage, cosmid, or virus in which another nucleic acid segment may be operably inserted so as to bring about the replication or expression of the segment. A “clone” is a population of cells derived from a single cell or common ancestor by mitosis. A “cell line” is a clone of a primary cell that is capable of stable growth in vitro for many generations. In some examples provided herein, cells are transformed by transfecting the cells with DNA.

The terms “express” and “produce” are used synonymously herein, and refer to the biosynthesis of a gene product. These terms encompass the transcription of a gene into RNA. These terms also encompass translation of RNA into one or more polypeptides, and further encompass all naturally occurring post-transcriptional and post-translational modifications.

The term “exogenous” as used herein, particularly in the context of cells or immune cells, refers to any material that is present and active in an individual living cell but that originated outside that cell (as opposed to an endogenous factor). The phrase “exogenous nucleic acid molecule” thus refers to a nucleic acid molecule that has been introduced in the (immune) cell, typically through transduction or transfection. The term “endogenous” as used herein refers to any factor or material that is present and active in an individual living cell and that originated from inside that cell (and that are thus typically also manufactured in a non-transduced or non-transfected cell).

A “promoter” as used herein is a regulatory region of nucleic acid usually located adjacent to a gene region, providing a control point for regulated gene transcription.

The term “subject” refers to human and non-human animals, including all vertebrates, e.g., mammals and non-mammals, such as non-human primates, mice, rabbits, sheep, dogs, cats, horses, cows, chickens, amphibians, and reptiles. In most particular embodiments of the described methods, the subject is a human.

The terms “treating” or “treatment” refer to any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, neurological examination, or psychiatric evaluations.

The phrase “adoptive cellular therapy”, “adoptive cell transfer”, or “ACT” as used herein refers to the transfer of cells, most typically immune cells, into a subject (e.g. a patient). These cells may have originated from the subject (in case of autologous therapy) or from another individual (in case of allogeneic therapy). The goal of the therapy is to improve immune functionality and characteristics, and in cancer immunotherapy, to raise an immune response against the cancer. Although T cells are most often used for ACT, it is also applied using other immune cell types such as NK cells, lymphocytes (e.g. tumor-infiltrating lymphocytes (TILs)), dendritic cells and myeloid cells.

An “effective amount” or “therapeutically effective amount” refers to an amount effective, at dosages and for periods of time necessary, to achieve a desired therapeutic result. A therapeutically effective amount of a therapeutic, such as the transformed immune cells described herein, may vary according to factors such as the disease state, age, sex, and weight of the individual, and the ability of the therapeutic (such as the cells) to elicit a desired response in the individual. A therapeutically effective amount is also one in which any toxic or detrimental effects of the therapeutic are outweighed by the therapeutically beneficial effects.

The phrase “graft versus host disease” or “GvHD” refers to a condition that might occur after an allogeneic transplant. In GvHD, the donated bone marrow, peripheral blood (stem) cells or other immune cells view the recipient's body as foreign, and the donated cells attack the body. As donor immunocompetent immune cells, such as T cells, are the main driver for GvHD, one strategy to prevent GvHD is by reducing (TCR-based) signaling in these immunocompetent cells, e.g. by directly or indirectly inhibiting the function of the TCR complex.

A “RNA interference molecule” as used herein is a molecule that mediates RNA interference (RNAi). Several mechanisms of RNAi gene modulation exist in plants and animals. A first is through the expression of small non-coding RNAs, called microRNAs (“miRNAs”). miRNAs are able to target specific messenger RNAs (“mRNA”) for degradation, and thereby promote gene silencing. Small interfering RNAs (“siRNAs”), which are artificially designed molecules, can also mediate RNAi. siRNAs can cause cleavage of a target molecule, such as mRNA, and similar to miRNAs, in order to recognize the target molecule, siRNAs rely on the complementarity of bases.

Within the class of molecules that are known as siRNAs are short hairpin RNAs (“shRNAs”). shRNAs are single stranded molecules that contain a sense region and an antisense region that is capable of hybridizing with the sense region. shRNAs are capable of forming a stem and loop structure in which the sense region and the antisense region form part or all of the stem. One advantage of using shRNAs is that they can be delivered or transcribed as a single molecule, which is not possible when an siRNA has two separate strands. However, like other siRNAs, shRNAs still target mRNA based on the complementarity of bases. A difference between shRNA molecules and miRNA molecules is that miRNA molecules are processed by Drosha, while conventional shRNA molecules are not (which has been associated with toxicity, Grimm et al., Nature 441:537-541 (2006)).

“CD52” as used herein refers to the product encoded by the CD52 gene (Gene ID: 1043 in humans), also known as Campath-1 antigen. CD52 is a glycoprotein present on the surface of mature lymphocytes, and comprises a peptide of 12 amino acids, anchored to glycosylphosphatidylinositol (GPI). It is highly negatively charged and presumably has an anti-adhesive function, allowing cells on which it is expressed to freely move around.

According to a first aspect, provided herein are engineered cells comprising:
A first exogenous nucleic acid molecule encoding a protein of interest; and
a second nucleic acid molecule encoding at least a RNA interference molecule directed against CD52.

The engineered cells are particularly eukaryotic cells, more particularly engineered mammalian cells, more particularly engineered human cells. According to particular embodiments, the cells are engineered immune cells. Typical immune cells are selected from T cells, NK cells, NKT cells, stem cells, progenitor cells, and iPSC cells.

According to particular embodiments, the engineered cells further contain a nucleic acid encoding a protein of interest. Proteins of interest can in principle be any protein, depending on the setting. However, typically they are proteins with a therapeutic function. These may include secreted therapeutic proteins, such as e.g. interleukins, cytokines or hormones. However, according to particular embodiments, the protein of interest is not secreted. Typically, the protein of interest is a receptor. According to further particular embodiments, the receptor is a chimeric antigen receptor or a TCR. Chimeric antigen receptors can be directed against any target expressed on the surface of a target cell, typical examples include, but are not limited to, CD5, CD19, CD20, CD22, CD23, CD30, CD33, CD38, CD44, CD56, CD70, CD123, CD133, CD138, CD171, CD174, CD248, CD274, CD276, CD279, CD319, CD326, CD340, BCMA, B7H3, B7H6, CEACAM5, EGFRvIII, EPHA2, mesothelin, NKG2D, HER2, HER3, GPC3, Flt3, DLL3, IL1RAP, KDR, MET, mucin 1, IL13Ra2, FOLH1, FAP, CA9, FOLR1, ROR1, GD2, PSCA, GPNMB, CSPG4, ULBP1, ULBP2, but many more exist and are also suitable. Although most CARs are scFv-based (i.e., the binding moiety is a scFv directed against a specific target, and the CAR is typically named after the target), some CARs are receptor-based (i.e., the binding moiety is part of a receptor, and the CAR typically is named after the receptor). An example of the latter is an NKG2D-CAR.

Engineered TCRs can be directed against any target of a cell, including intracellular targets. In addition to the above listed targets present on a cell surface, typical targets for a TCR include, but are not limited to, NY-ESO-1, PRAME, AFP, MAGE-A3, MAGE-A4, MAGE-A6, MAGE-A10, MAGE-A12, gp100, MART-1, tyrosinase, WT1, p53, HPV-E6, HPV-E7, HBV, TRAIL, thyroglobulin, KRAS, HERV-E, HA-1, CMV, and CEA.

According to these particular embodiments where a further protein of interest is present, the first and second nucleic acid molecule in the engineered cell are typically present in one vector, such as a eukaryotic expression plasmid, a mini-circle DNA, or a viral vector (e.g. derived from a lentivirus, a retrovirus, an adenovirus, an adeno-associated virus, and a Sendai virus).

The engineered cells are particularly eukaryotic cells, more particularly engineered mammalian cells, more particularly engineered human cells. According to particular embodiments, the cells are engineered immune cells. Typical immune cells are selected from a T cell, a NK cell, a NKT cell, a stem cell, a progenitor cell, and an iPSC cell.

According to particular embodiments, the RNA interference molecules can be shRNA molecules or miRNA molecules. Most particularly, they are miRNA molecules. Particularly suited scaffold sequences for miRNA molecules are a miR-30 scaffold sequence, a miR-155 scaffold sequence, and a miR-196a2 scaffold sequence. Typically, the miRNA molecule comprises a miR-196 scaffold sequence, preferably a miR-196a2 scaffold sequence.

Further suitable scaffold sequences include miR-26b (hsa-mir-26b), miR-204 (hsa-mir-204), and miR-126 (hsa-mir-126), hsa-let-7f, hsa-let-7g, hsa-let-7a, hsa-let-7b, hsa-let-7c, hsa-mir-29a, hsa-mir-140-3p, hsa-let-7i, hsa-let-7e, hsa-mir-7-1, hsa-mir-7-2, hsa-mir-7-3, hsa-mir-26a, hsa-mir-26a, hsa-mir-340, hsa-mir-101, hsa-mir-29c, hsa-mir-191, hsa-mir-222, hsa-mir-34c-5p, hsa-mir-21, hsa-mir-378, hsa-mir-100, hsa-mir-192, hsa-mir-30d, hsa-mir-16, hsa-mir-432, hsa-mir-744, hsa-mir-29b, hsa-mir-130a, or hsa-mir-15a.

According to particular embodiments, the at least one RNA interference molecule is under control of a promoter. According to particular embodiments, the first and second nucleic acid molecules are under control of the same promoter. According to specific embodiments, the promoter is selected from a Pol II promoter, and a Pol III promoter. According to particular embodiments, the promoter is a natural or synthetic Pol II promoter. Suitable promoters include, but are not limited to, a cytomegalovirus (CMV) promoter, an elongation factor 1 alpha (EF1α) promoter (core or full length), a phosphoglycerate kinase (PGK) promoter, a composite beta-actin promoter with an upstream CMV IV enhancer (CAG promoter), a ubiquitin C (UbC) promoter, a spleen focus forming virus (SFFV) promoter, a Rous sarcoma virus (RSV) promoter, an interleukin-2 promoter, a murine stem cell virus (MSCV) long terminal repeat (LTR), a Gibbon ape leukemia virus (GALV) LTR, a simian virus 40 (SV40) promoter, and a tRNA promoter. These promoters are among the most commonly used polymerase II promoters to drive mRNA expression.

According to specific embodiments, the promoter is an inducible promoter. This allows temporal regulation of expression, and can be particularly valuable in case CD52 expression no longer needs to be suppressed (e.g. to increase mobility; or in case the anti-CD52 therapy is no longer present).

According to further particularly envisaged embodiments, cells are provided which have been further engineered to reduce or inactivate TCR signalling. This can e.g. be achieved by functionally impairing or reducing expression of the endogenous T cell receptor (TCR), as outlined in e.g. U.S. Pat. No. 9,181,527.

These embodiments are particularly envisaged, as anti-CD52 lymphodepletion is primarily (but not solely) envisaged for allogeneic therapy. In allogeneic therapy, endogenous TCR signalling will indeed be reduced or inactivated. Inactivation of TCR can e.g. be achieved through gene editing (e.g. using Crispr/CAS, TALEN, ZFN, Meganucleases or MegaTAL technology). Typical ways of reducing TCR signalling is through expression of dominant negative proteins (e.g. TIM proteins as described in U.S. Pat. No. 9,181,527), or by using RNA interference. RNA interference to reduce TCR signalling will typically entail the use of a RNA interference molecule directed against a TCR receptor complex subunit (e.g. against TCR receptor α chain, TCR β chain, CD3γ chain, CD3δ chain, CD3ε chain, CD3ζ-chain (aka CD247)).

As is the case for the CD52 RNA interference molecules, RNA interference molecules against TCR receptor complex can be shRNA molecules or miRNA molecules. Most particularly, they are miRNA molecules. Particularly suited scaffold sequences for miRNA molecules are a miR-30 scaffold sequence, a miR-155 scaffold sequence, and a miR-196a2 scaffold sequence. Typically, the miRNA molecule comprises a miR-196 scaffold sequence, preferably a miR-196a2 scaffold sequence. However, other scaffolds can be used as well (as described previously for CD52).

According to a further aspect, methods are provided herein of generating the engineered cells described. These typically entail the isolation of cells suitable for adoptive cell therapy, and transduction with
A first exogenous nucleic acid molecule encoding a protein of interest; and
a second nucleic acid molecule encoding at least a RNA interference molecule directed against CD52.

The features of these cells are as outlined above.

According to a further aspect, the engineered cells described herein are provided for use as a medicament. According to specific embodiments, the engineered cells are provided for use in the treatment of cancer. Exemplary types of cancer that can be treated include, but not limited to, adenocarcinoma, adrenocortical carcinoma, anal cancer, astrocytoma, bladder cancer, bone cancer, brain cancer, breast cancer, cervical cancer, colorectal cancer, endometrial cancer, esophageal cancer, Ewing sarcoma, eye cancer, Fallopian tube cancer, gastric cancer, glioblastoma, head and neck cancer, Kaposi sarcoma, kidney cancer, leukemia, liver cancer, lung cancer, lymphoma, melanoma, mesothelioma, myelodysplastic syndrome, multiple myeloma, neuroblastoma, osteosarcoma, ovarian cancer, pancreatic cancer, parathyroid cancer, penile cancer, peritoneal cancer, pharyngeal cancer, prostate cancer, renal cell carcinoma, retinoblastoma, rhabdomyosarcoma, sarcoma, skin cancer, small intestine cancer, stomach cancer, testicular cancer, thyroid cancer, urethral cancer, uterine cancer, vaginal cancer, and Wilms tumor.

According to particular embodiments, the cells can be provided for treatment of liquid or blood cancers. Examples of such cancers include e.g. leukemia (including a.o. acute myelogenous leukemia (AML), acute lymphocytic leukemia (ALL), chronic myelogenous leukemia (CML), and chronic lymphocytic leukemia (CLL)), lymphoma (including a.o. Hodgkin's lymphoma and non-Hodgkin's lymphoma such as B-cell lymphoma (e.g. DLBCL), T cell lymphoma, Burkitt's lymphoma, follicular lymphoma, mantle cell lymphoma, and small lymphocytic lymphoma), multiple myeloma or myelodysplastic syndrome (MDS).

This is equivalent as saying that methods of treating cancer are provided, comprising administering to a subject in need thereof a suitable dose of engineered cells as described herein (i.e. engineered cells comprising a first exogenous nucleic acid molecule encoding at least a RNA interference molecule against CD52), thereby improving at least one symptom associated with the cancer. Cancers envisaged for treatment include, but are not limited to, adenocarcinoma, adrenocortical carcinoma, anal cancer, astrocytoma, bladder cancer, bone cancer, brain cancer, breast cancer, cervical cancer, colorectal cancer, endometrial cancer, esophageal cancer, Ewing sarcoma, eye cancer, Fallopian tube cancer, gastric cancer, glioblastoma, head and neck cancer, Kaposi sarcoma, kidney cancer, leukemia, liver cancer, lung cancer, lymphoma, melanoma, mesothelioma, myelodysplastic syndrome, multiple myeloma, neuroblastoma, osteosarcoma, ovarian cancer, pancreatic cancer, parathyroid cancer, penile cancer, peritoneal cancer, pharyngeal cancer, prostate cancer, renal cell carcinoma, retinoblastoma, rhabdomyosarcoma, sarcoma, skin cancer, small intestine cancer, stomach cancer, testicular cancer, thyroid cancer, urethral cancer, uterine cancer, vaginal cancer, and Wilms tumor. According to further particular embodiments, methods of treating blood cancer are provided, comprising administering to a subject in need thereof a suitable dose of engineered cells as described herein thereby improving at least one symptom of the cancer.

Most particularly, the subjects being treated will be subjects that also receive a lymphodepletion regimen comprising an anti-CD52 antibody treatment (e.g. alemtuzumab). Typically, the lymphodepletion regimen will be administered prior to the infusion of the engineered cells, although concomitant or concurrent administration is also possible. Most typically, the presence of the CD52 antibody will in the subject will at least partially overlap in time with the presence of the engineered cells that are administered.

The engineered cells may be autologous immune cells (cells obtained from the patient) or allogeneic immune cells (cells obtained from another subject), but most typically, they are allogeneic cells.

According to alternative embodiments, the cells can be provided for use in the treatment of autoimmune disease. Exemplary types of autoimmune diseases that can be treated include, but are not limited to, rheumatoid arthritis (RA), systemic lupus erythematosus (SLE), inflammatory bowel disease (IBD), multiple sclerosis (MS), Type 1 diabetes mellitus, amyotrophic lateral sclerosis (ALS or Lou Gehrig's disease), spinal muscular atrophy (SMA), Crohn's disease, Guillain-Barre syndrome, chronic inflammatory demyelinating polyneuropathy, psoriasis, psoriatic arthritis, Addison's disease, ankylosing spondylitis, Behcet's disease, coeliac disease, Coxsackie myocarditis, endometriosis, fibromyalgia, Graves' disease, Hashimoto's thyroiditis, Kawasaki disease, Meniere's disease, myasthenia gravis, sarcoidosis, scleroderma, Sjögren's syndrome, thrombocytopenic purpura (TTP), ulcerative colitis, vasculitis and vitiligo.

This is equivalent as saying that methods of treating autoimmune disease are provided, comprising administering to a subject in need thereof a suitable dose of engineered cells as described herein, thereby improving at least one symptom associated with the autoimmune disease. Exemplary autoimmune diseases that can be treated are listed above.

According to yet further embodiments, the cells can be provided for use in the treatment of infectious disease. “Infectious disease” is used herein to refer to any type of disease caused by the presence of an external organism (pathogen) in or on the subject or organism with the disease. Infections are usually considered to be caused by microorganisms or microparasites like viruses, prions, bacteria, and viroids, though larger organisms like macroparasites and fungi can also infect. The organisms that can cause infection are herein referred to as “pathogens” (in case they cause disease) and “parasites” (in case they benefit at the expense of the host organism, thereby reducing biological fitness of the host organism, even without overt disease being present) and include, but are not limited to, viruses, bacteria, fungi, protists (e.g. Plasmodium) and protozoa (e.g. Plasmodium, Entamoeba, Giardia, Toxoplasma, Cryptosporidium, Trichomonas, Leishmania, Trypanosoma) (microparasites) and macroparasites such as worms (e.g. nematodes like ascarids, filarias, hookworms, pinworms and whipworms or flatworms like tapeworms and flukes), but also ectoparasites such as ticks and mites. Parasitoids, i.e. parasitic organisms that sterilize or kill the host organism, are envisaged within the term parasites. According to particular embodiments, the infectious disease is caused by a microbial or viral organism.

“Microbial organism,” as used herein, may refer to bacteria, such as gram-positive bacteria (eg, Staphylococcus sp., Enterococcus sp., Bacillus sp.), Gram-negative bacteria. (for example, Escherichia sp., Yersinia sp.), spirochetes (for example, Treponema sp, such as Treponema pallidum, Leptospira sp., Borrelia sp., such as Borrelia burgdorferi), mollicutes (i.e. bacteria without cell wall, such as Mycoplasma sp.), acid-resistant bacteria (for example, Mycobacterium sp., such as Mycobacterium tuberculosum, Nocardia sp.). “Microbacterial organisms” also encompass fungi (such as yeasts and molds, for example, Candida sp., Aspergillus sp., Coccidioides sp., Cryptococcus sp., Histoplasma sp., Pneumocystis sp. Or Trichophyton sp.), Protozoa (for example, Plasmodium sp., Entamoeba sp., Giardia sp., Toxoplasma sp., Cryptosporidium sp., Trichomonas sp., Leishmania sp., Trypanosoma sp.) and archaea. Further examples of microbial organisms causing infectious disease that can be treated with the instant methods include, but are not limited to, Staphylococcus aureus (including methicillin-resistant S. aureus (MRSA)), Enterococcus sp. (including vancomycin-resistant enterococci (VRE), the nosocomial pathogen Enterococcus faecalis), food pathogens such as Bacillus subtilis, B. cereus, Listeria monocytogenes, Salmonella sp., and Legionella pneumophilia.

“Viral organism” or “virus”, which are used as equivalents herein, are small infectious agents that can replicate only inside the living cells of organisms. They include dsDNA viruses (e.g. Adenoviruses, Herpesviruses, Poxviruses), ssDNA viruses (e.g. Parvoviruses), dsRNA viruses (e.g. Reoviruses), (+)ssRNA viruses (e.g. Picornaviruses, Togaviruses, Coronaviruses), (−)ssRNA viruses (e.g. Orthomyxoviruses, Rhabdoviruses), ssRNA-RT (reverse transcribing) viruses, i.e. viruses with (+) sense RNA with DNA intermediate in life-cycle (e.g. Retroviruses), and dsDNA-RT viruses (e.g. Hepadnaviruses). Examples of viruses that can also infect human subjects include, but are not limited to, an adenovirus, an astrovirus, a hepadnavirus (e.g. hepatitis B virus), a herpesvirus (e.g. herpes simplex virus type I, the herpes simplex virus type 2, a Human cytomegalovirus, an Epstein-Barr virus, a varicella zoster virus, a roseolovirus), a papovavirus (e.g. the virus of human papilloma and a human polyoma virus), a poxvirus (e.g. a variola virus, a vaccinia virus, a smallpox virus), an arenavirus, a buniavirus, a calcivirus, a coronavirus (e.g. SARS coronavirus, MERS coronavirus, SARS-CoV-2 coronavirus (etiologic agent of COVID-19)), a filovirus (e.g. Ebola virus, Marburg virus), a flavivirus (e.g. yellow fever virus, a western Nile virus, a dengue fever virus, a hepatitis C virus, a tick-borne encephalitis virus, a Japanese encephalitis virus, an encephalitis virus), an orthomyxovirus (e.g. type A influenza virus, type B influenza virus and type C influenza virus), a paramyxovirus (e.g. a parainfluenza virus, a rubulavirus (mumps), a morbilivirus (measles), a pneumovirus, such as a human respiratory syncytial virus), a picornavirus (e.g. a poliovirus, a rhinovirus, a coxackie A virus, a coxackie B virus, a hepatitis A virus, an ecovirus and an enterovirus), a reovirus, a retrovirus (e.g. a lentivirus, such as a human immunodeficiency virus and a human T lymphotrophic virus (HTLV)), a rhabdovirus (e.g. rabies virus) or a togavirus (e.g. rubella virus).

This is equivalent as saying that methods of treating infectious disease are provided, comprising administering to a subject in need thereof a suitable dose of engineered cells as described herein, thereby improving at least one symptom. Particularly envisaged microbial or viral infectious diseases are those caused by the pathogens listed above.

These cells that are provided for use as a medicament can be provided for use in allogeneic therapies. I.e., they are provided for use in treatments where allogeneic ACT is considered a therapeutic option (wherein cells from another subject are provided to a subject in need thereof). According to specific embodiments, in allogeneic therapies, in addition to the protein of interest and the RNA interference molecule against CD52, the cells will further be engineered to have reduced functional TCR expression (e.g. by genetic knockout, or by expression of an additional molecule, such as a RNA interference molecule, directed against the TCR (most particularly, against a subunit of the TCR complex)). According to alternative embodiments, these cells are provided for use in autologous therapies, particularly autologies ACT therapies (i.e., with cells obtained from the patient).

It is to be understood that although particular embodiments, specific configurations as well as materials and/or molecules, have been discussed herein for cells and methods according to present invention, various changes or modifications in form and detail may be made without departing from the scope and spirit of this invention. The following examples are provided to better illustrate particular embodiments, and they should not be considered limiting the application. The application is limited only by the claims.

### EXAMPLES

### Example 1. Screening of Different CD52 Targeting shRNAs

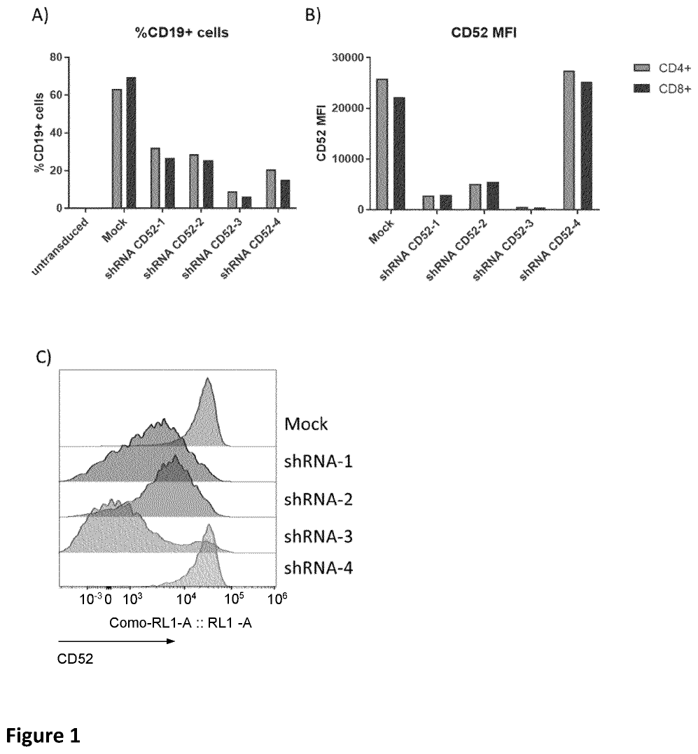

To be able to compare the efficiency of different CD52 targeting shRNAs, an identical backbone construct was used, where only the CD52 targeting sequence of the shRNA was exchanged. A retroviral vector was used to deliver the shRNAs into primary T cells, which could be tracked by a truncated CD19 (tCD19) marker. Initially, four different CD52-targeting shRNAs were cloned into the retroviral vector encoding a tCD19 reporter. The degree of CD52 knockdown was assessed at harvest at day 8 of the process. All constructs were able to transduce primary T cells, as measured by CD19-positivity (FIG. 1). However, the degree of CD52 knockdown was different for the 4 shRNAs tested. shRNA-3 was most efficient at knocking down CD52 expression, followed by shRNA-1 and shRNA-2. On the other hand, shRNA-4 showed no decrease in CD52 expression (FIG. 1).

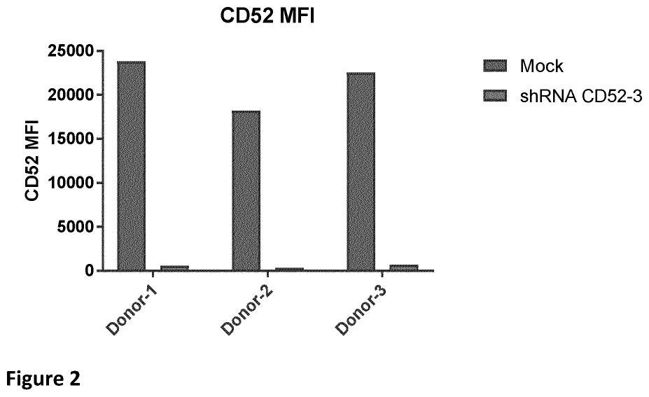

In order to assess whether knockdown efficiency of this shRNA would vary between different donors, T cells from three different donors were transduced with a Mock construct or a CD52 shRNA-3 expression construct (FIG. 2). In all three donors the shRNA-3 showed a significant and consisted knockdown of CD52 (FIG. 2). Thus, the identified shRNA results in consistent knockdown of CD52.

### Example 2. Screening of gRNAs for CRISPR/Cas9 Mediated CD52 Knockout

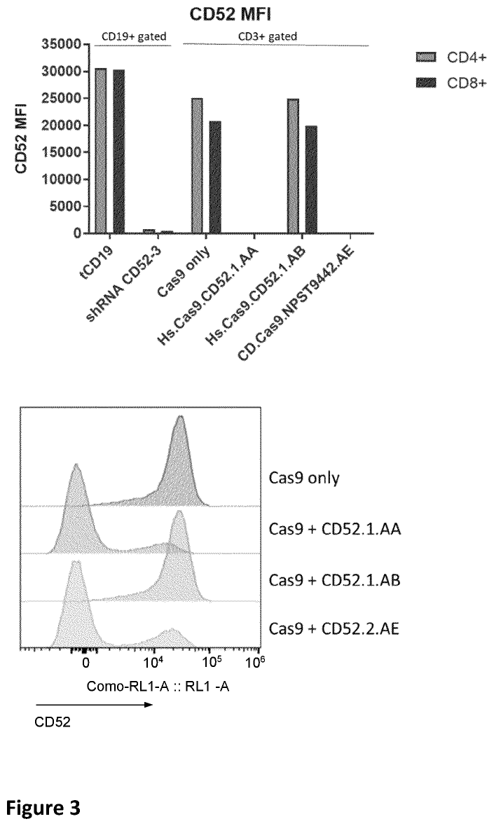

Next, we aimed to develop a method for generating a CD52 knockout T cell by use of genome editing technology. To this end, different guide RNAs (gRNAs) were designed to identify the ones that would efficiently generate CD52-deficient T cells. The different gRNAs were assessed for their potential to generate CD52-knockout T cells (FIG. 3). Two out of three gRNAs were able to generate CD52 knockout cells (gRNA-1 and gRNA-3), whereas the frequency of CD52-deficient cells was slightly higher with CD52 gRNA-1.

### Example 3. Co-Expression of CD52 shRNA and a BCMA-Specific Chimeric Antigen Receptor (CAR)

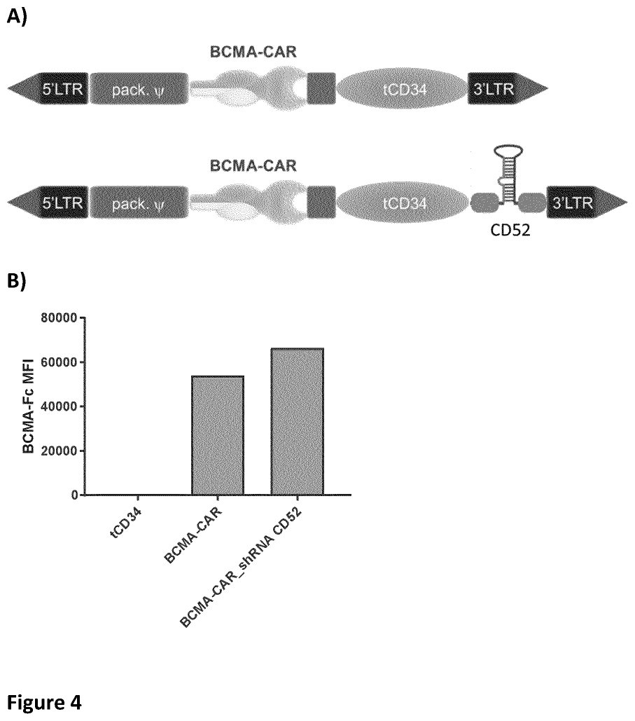

We next aimed to combine the CD52 shRNA with a chimeric antigen receptor (CAR). To this end, an all-in-one vector was designed. FIG. 4A shows the design of a classical CAR expression vector, based on a retroviral vector and further contains a selectable marker. Further, for use in adoptive cell therapy setting, an identical construct was made that further contained shRNA against CD52 (FIG. 4A, bottom). When expressed in cells, this shRNA interferes with the expression of CD52 and thus can protect the cells from treatment with an anti-CD52 targeting antibody (e.g. Alemtuzumab) in ACT.

The effect of the co-expression of a CD52-targeting shRNA on the expression of a BCMA CAR was assessed by staining with BCMA-Fc fusion protein. In a second step, cells were stained with PE-conjugated anti-Fc and an APC-conjugated anti-CD34 antibodies. FIG. 4B shows the median florescence intensity for the BCMA-Fc staining for cells transduced with a tCD34 control vector, a BCMA-CAR and a BCMA-CAR co-expressing the CD52 shRNA.

### Example 4. Activity Against Tumor Cells

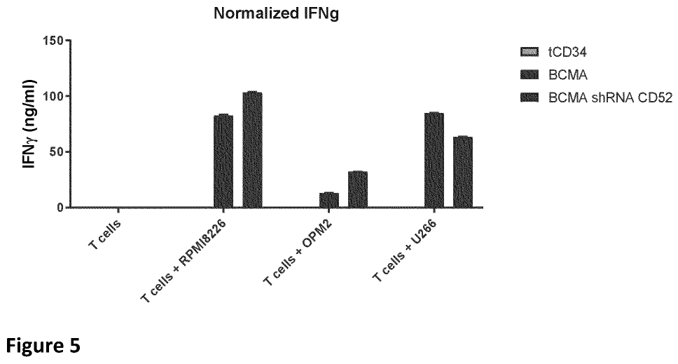

To further evaluate the activity of different anti-BCMA CARs with or without the CD52 targeting shRNA, cellular activity of the different BCMA-CAR T cells was assessed by co-culturing the cells with different BCMA-positive cancer cell lines (RPMI-8226, U266, OPM-2). Interferon gamma secretion was used to measure activity against cancer cells. To this end, different BCMA expressing cancer cell lines were co-cultured with Mock (tCD34), or BCMA-CAR expressing T cells or T cells co-expressing the BCMA-CAR and a CD52 targeting shRNA. 24 h after co-culture, IFN-γ levels were measured in the supernatants. Results are shown in FIG. 5. These experiments show that the co-expression of the shRNA does not affect the activity of the CAR.

### Example 5. In Vivo Activity Against Tumor

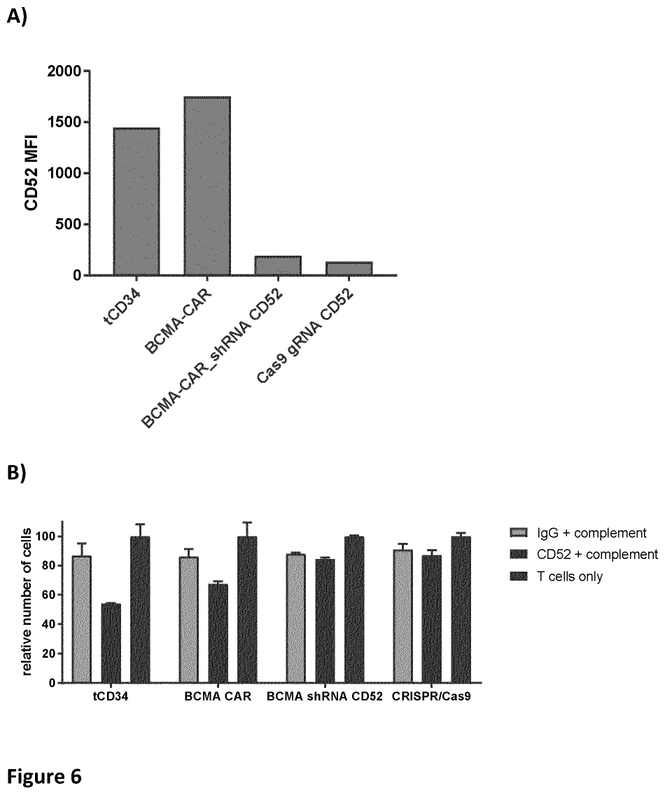

The goal of CD52 downmodulation is to generate T cells that are resistant to anti-CD52 antibody mediated immunosuppression. T cells were transduced with either a Mock (tCD34), a BCMA-CAR or an BCMA-CAR co-expressing a CD52 targeting shRNA. As a control, T cells were nucleofected with a ribonucleoprotein (RNP) complex of a CD52 gRNA and Cas9 protein. CD52 expression is shown in FIG. 6A for the different conditions.

Cells were treated with 50 μg/ml anti-CD52 monoclonal antibody or rat IgG as control. In order to induce complement mediated cytotoxicity, 20% rabbit complement was added to the culture. After four hours of incubation, cells were counted by flow cytometry to measure complement mediated cytotoxicity. FIG. 6B shows the results of this assay. Cell counts were normalized to counts of T cells cultured in the absence of antibody and complement. It can be clearly seen that shRNA mediated knockdown protects as well as CRISPR against CD52 antibody-mediated cytotoxicity.

### Example 6. Generation of Allogeneic T Cells Expressing CD52 shRNA

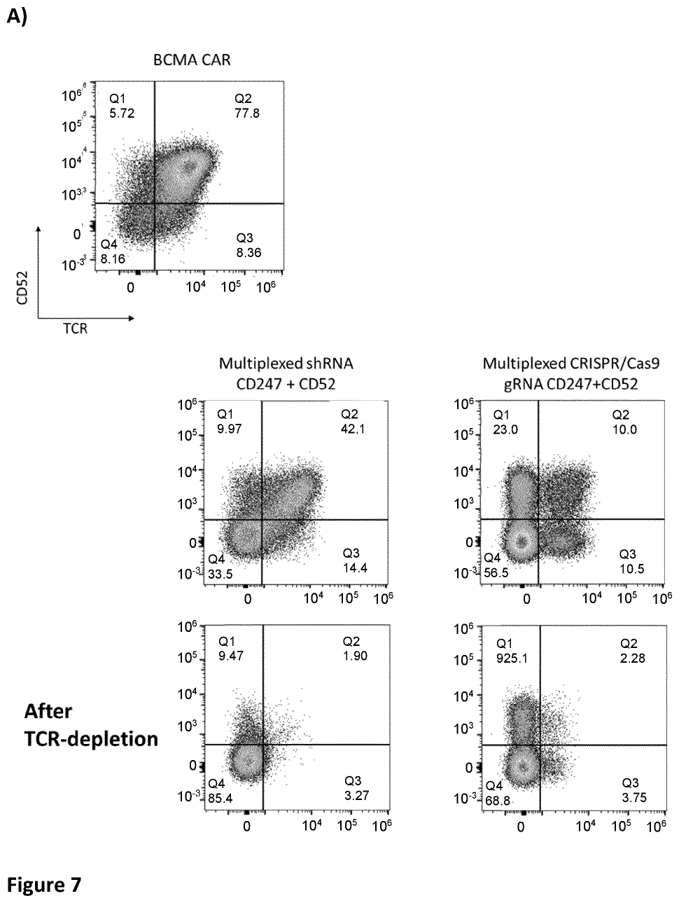

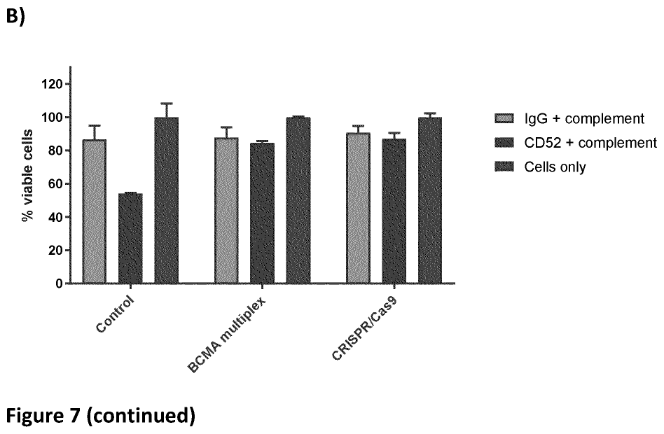

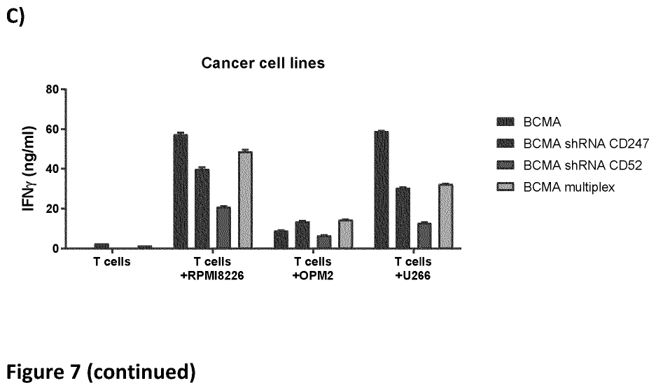

To allow the cells to be used in an allogeneic setting, shRNA targeting the CD3zeta chain and targeting CD52 were co-expressed in T cells. The dual shRNA expression allows isolation of CAR T cell populations with multiple protein knock down with a single purification step. FIG. 7A compares TCR and CD52 expression after shRNA knockdown or CRISPR knockout respectively. With CRISPR, a population remains that has no T cell but expresses CD52, in addition to the double knockout. With the two shRNAs, only one population, with no or low CD52 and TCR, remains.

To further compare the phenotype of these T cells with dual knockdown of CD3 zeta and CD52 to the phenotype and anti-tumor activity as the ones with only CD52 knockdown, the same experiments were done. Briefly, as shown in FIG. 7B, cells were treated with 50 μg/ml anti-CD52 monoclonal antibody or rat IgG as control. In order to induce complement mediated cytotoxicity, 20% rabbit complement was added to the culture. After four hours of incubation, cells were counted by flow cytometry to measure complement mediated cytotoxicity. Cell counts were normalized to counts of T cells cultured in the absence of antibody and complement. It can be clearly seen that the double shRNA mediated knockdown protects as well as CRISPR against CD52 antibody-mediated cytotoxicity. These results are also comparable to the single knockdown of CD52 shown in FIG. 6B.

As shown in FIG. 7C, CAR efficacy was assessed in co-culture with different cancer cell lines. The following CAR T cells were assessed side by side: BCMA, BCMA with shRNA against CD247, BCMA CAR with shRNA against CD52, BCMA multiplex (with both shRNA against CD247 and CD52). These results show that cancer cells, but not T cells alone, led to CAR activation. The CAR with multiplex shRNA is as effective as the CAR with single shRNA.

1. An engineered cell comprising:
A first exogenous nucleic acid molecule encoding a protein of interest
a second nucleic acid molecule encoding at least a RNA interference molecule directed against CD52.

2. The engineered cell of claim 1, which is an engineered immune cell.

3. The engineered immune cell of claim 1 or 2, wherein the immune cell is selected from a T cell, a NK cell, a NKT cell, a stem cell, a progenitor cell, and an iPSC cell.

4. The engineered cell of any one of claims 1 to 3, wherein the protein of interest is a receptor, particularly a chimeric antigen receptor or a TCR.

5. The engineered cell of any one of claims 1 to 4, wherein the first and second nucleic acid molecule are present in one vector, such as a eukaryotic expression plasmid, a mini-circle DNA, or a viral vector (e.g. derived from a lentivirus, a retrovirus, an adenovirus, an adeno-associated virus, and a Sendai virus).

6. The engineered cell of any one of claims 1 to 5, wherein the RNA interference molecule is a shRNA molecule.

7. The engineered cell of claim 6, wherein the RNA interference molecule is a miRNA molecule.

8. The engineered cell of claim 7, wherein the miRNA molecule comprises a miR-196 scaffold sequence, preferably a miR-196a2 scaffold sequence.

9. The engineered cell of any one of claims 1 to 8, wherein the RNA interference molecule is under control of a promoter selected from a cytomegalovirus (CMV) promoter, an elongation factor 1 alpha (EF1α) promoter, a phosphoglycerate kinase (PGK) promoter, a composite beta-actin promoter with an upstream CMV IV enhancer (CAG promoter), a ubiquitin C (UbC) promoter, a spleen focus forming virus (SFFV) promoter, a Rous sarcoma virus (RSV) promoter, an interleukin-2 promoter, a murine stem cell virus (MSCV) long terminal repeat (LTR), a Gibbon ape leukemia virus (GALV) LTR, a simian virus 40 (SV40) promoter, and a tRNA promoter.

10. The engineered cell of claim 9, wherein the promoter is inducible.

11. The engineered cell of any one of claims 1 to 10, which has been further engineered to reduce or inactivate TCR signalling.

12. The engineered cell of claim 11, wherein TCR signalling has been inactivated through gene editing.

13. The engineered cell of claim 12, wherein TCR signalling has been reduced through RNA interference.

14. The engineered cell of claim 13, wherein the RNA interference to reduce TCR signalling comprises a RNA interference molecule directed against a TCR receptor complex subunit.

15. The engineered cell of any one of claims 1 to 14 for use as a medicament.

16. The engineered cell of any one of claims 1 to 14 for use in the treatment of cancer.

17. A method of treating cancer, comprising administering to a subject in need thereof a suitable dose of cells according to any one of claims 1 to 14, thereby improving at least one symptom.
